(19) United States
(12) Patent Application Publication
(10) Pub. No.: US 2014/0060548 A1
(43) Pub. Date: Mar. 6, 2014

(54) Protective Bandage Device

(71) Applicant: Todd Check, Indianapolis, IN (US)

(72) Inventor: Todd Check, Indianapolis, IN (US)

(21) Appl. No.: 14/116,496

(22) PCT Filed: May 14, 2012
(86) PCT No.: PCT/US2012/037707

§ 371 Date: Nov. 8, 2013

Related U.S. Application Data
(60) Provisional application No. 61/486,882, filed on May 17, 2011.

(51) Int. Cl. A61F 15/00

(52) U.S. Cl. CPC A61F 15/008
USPC 128/845

(57) ABSTRACT

The present invention relates to a protective bandage device and to methods of use thereof.

This application claims priority from U.S. Provisional Patent Application Ser. No. 61/486,882 filed May 17, 2011, the entire contents of which is hereby incorporated by reference.

### FIELD OF THE INVENTION

The present invention relates generally to a protective bandage for use by a person in need thereof wherein said bandage covers but does not contact or touch a wound or ancillary medical device, and to methods of use thereof. More particularly, the protective bandage of the invention attaches to a person to form a protective shield over and around a wound or ancillary medical device.

### BACKGROUND OF THE INVENTION

Currently, there exists a number of conventional first-aid bandages including, for example, contour bandages, adhesive bandages, surgical bandages, and gauze bandages that apply evenly and smoothly over flat, contoured, and/or flexing areas of the body such as elbows, knees, heels, torso, legs, arms, etc. See, for example, U.S. Pat. Nos. D611,156, 7,605,299, and 7,176,343. These types of bandages are generally designed to provide self-adhesive tabs plus a centrally located absorbent pad. An adhesive backing is provided consisting of a flexible, breathable, gas-permeable, hydrophobic material. The adhesive backing is typically a woven or nonwoven plastic, paper, or other suitably soft material.

Unfortunately, the soft material provided for the backing or the absorbent pad of bandages of this type do not protect the wound from contact by forces and/or objects in the everyday environment. For example, persons who play sports frequently injure their knee, elbow, or other body part during the activity. After a wound is cleansed, a contour bandage or a general-purpose bandage may be applied to the wound. Alternatively, the wounded person may not apply a bandage to the wound thereby leaving the wound exposed to the environment. Whether a bandage is applied or not, the person may encounter disturbing and/or painful insults to the wound from the every day environment. For example, when the person sleeps and rolls over onto the wound pressure may be felt on the wound thereby causing pain. Additionally, as the person resumes normal activity, the person may accidentally bump the healing wound against an object thereby causing additional pain. Moreover, during the healing process, the wound will often seep blood or other liquid. If a conventional bandage is applied to the wound, then the blood or liquid is absorbed by the absorbent pad of the bandage. Often the absorbent pad filled with blood dries and sticks to the wound causing pain for the person when the bandage is removed. If no bandage is applied to the wound, thereby leaving the wound open, then blood seeping out of the wound will cause the wound to stick to clothing or bed sheets as the blood dries.

Thus, there remains a need for improved protective bandages in this field.

### SUMMARY OF THE INVENTION

This summary is provided merely to introduce certain concepts and is not intended to identify any key or essential features of the claimed subject matter.

Certain variations of the invention provide improved bandage devices for medical use including but not limited to wound protection and protection of ancillary medical devices attached to a patient.

Accordingly, it is an object of the present invention to provide a medically useful bandage protection device for use by a patient or individual in need thereof.

Another object of the present invention is to provide methods for facilitating wound healing.

Another object of the present invention is to provide methods for facilitating medical or veterinary care or treatment that utilizes ancillary medical devices including but not limited to tubes, catheters, stents, syringes, and splints.

### BRIEF DESCRIPTION OF THE DRAWINGS

FIG. 1a is a perspective view of a first embodiment of a dome-shaped protective bandage device.

FIG. 1b is a side view of the first embodiment shown in FIG. 1a.

FIG. 1c is a bottom view of the first embodiment depicted in FIG. 1a.

FIG. 1d is a side cut-away view of a variation on the dome-shaped bandage device depicted in FIG. 1a.

FIG. 1e is a top view of the variation embodiment depicted in FIG. 1d.

FIG. 1f is a perspective view of an embodiment of a dome-shaped protective bandage having a hole or outlet to accommodate the passage of tube(s).

FIG. 2 is a side view of a second embodiment of a bowl-shaped protective bandage device.

FIG. 3a is a perspective view of a rectangular arch-shaped third embodiment of a protective bandage device.

FIG. 3b is a partial cross-sectional view of one embodiment of a male-female junction between two rectangular arch-shaped bandage devices.

### DESCRIPTION OF THE SELECTED EMBODIMENTS

As used herein the term “ancillary medical device” refers to any medical apparatus or device that is incorporated, applied, or inserted onto or within the body of a patient for purposes including treating any chronic or acute disease or condition. Exemplary but non exclusive ancillary medical devices include catheters, stents, hypodermic syringe needles, tubes, fluid drainage tubes, feeding tubes, splints, etc.

As used herein, the term “sloping” or “sloped” refers generally to an aspect of the configuration of a wall of a bandage device of the present invention wherein the angle between the surface of the wall at or near the outer edge of the device is other than perpendicular; generally and preferably the sloped angle is less than 90°.

As used herein the term “wound” is intended to broadly include any insult, injury, or condition of the body that may or may not cause pain, tissue damage, tissue degeneration, or malfunction, including wounds leading to blood loss, whether intentionally inflicted for medial reasons, e.g. surgery, or having arisen by accident, violence, or during sports, or as a result of disease, including but not limited to bruises, cuts, abrasions, scrapes, surgical incisions, strains, surgical stitches, sprains, fractures, or degenerative diseases such as, for example, arthritis, inflammatory disease, fibromyalgia, lupus, cancer, etc.

As used herein, the terms “person”, “patient”, and “individual” all refer to a mammal including animals and humans on which a device of the present invention can be used.

In one aspect, a device according to the present invention is provided as a means to foster or promote healing of a wound when used alone or in combination with other medical treatments and/or devices including but not limited to band-aids, bandages or other medical treatments including but not limited to the ingestion or application of medicaments including orally or topically administered medicaments or pharmaceuticals such as antiseptics, antifungal agents, antiviral agents, anti-inflammatory agents, pain relievers, etc.

A device of the invention provides a protective barrier or shield for a wound or ancillary medical device against undesired environmental insults such as bumps, pressure or other undesired contact that can cause pain or interrupt the healing process or in the case of ancillary medical devices, disrupt or dislodge such devices from a patient thereby impeding the clinical benefit intended for such devices. The bandage devices of the present invention do not include an absorbent pad(s). In one aspect, a device of the invention can be used in conjunction with ancillary medical devices to provide protection for such devices, for example, from dislodgement from the body. As such, users of a device according to the present invention are able to foster healing and avoid additional discomfort or pain caused by such undesired environmental insults. In addition, the healing process is allowed to proceed without interruption as, for example, when an incipient scab is dislodged by an inadvertent force encountered in the environment.

For the purpose of promoting an understanding of the principles of the invention, reference will now be made to certain aspects of the embodiments of the invention as illustrated in the drawings and disclosure herein. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

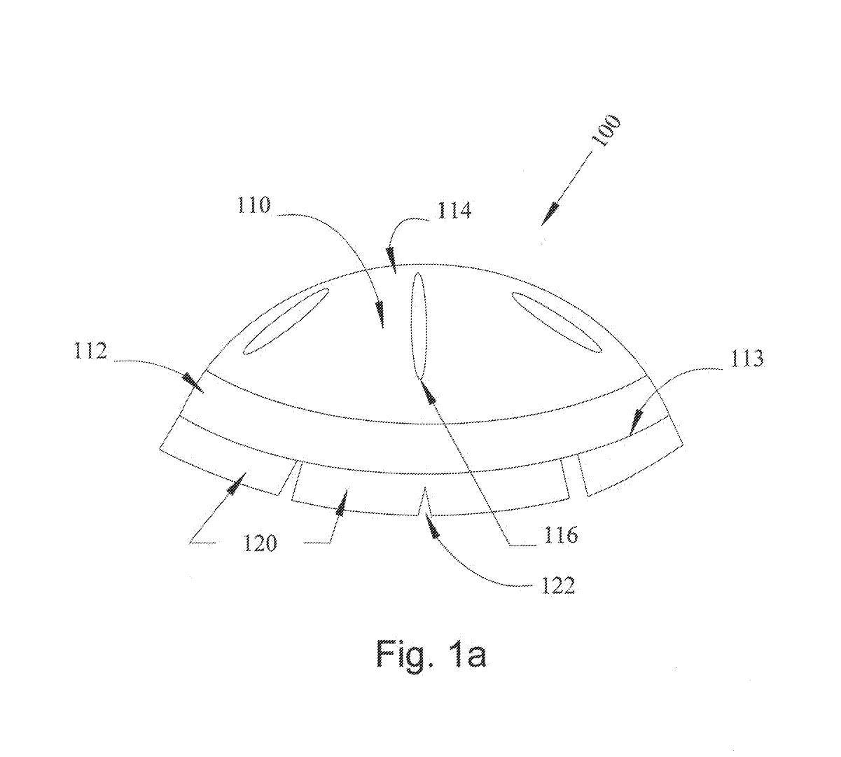

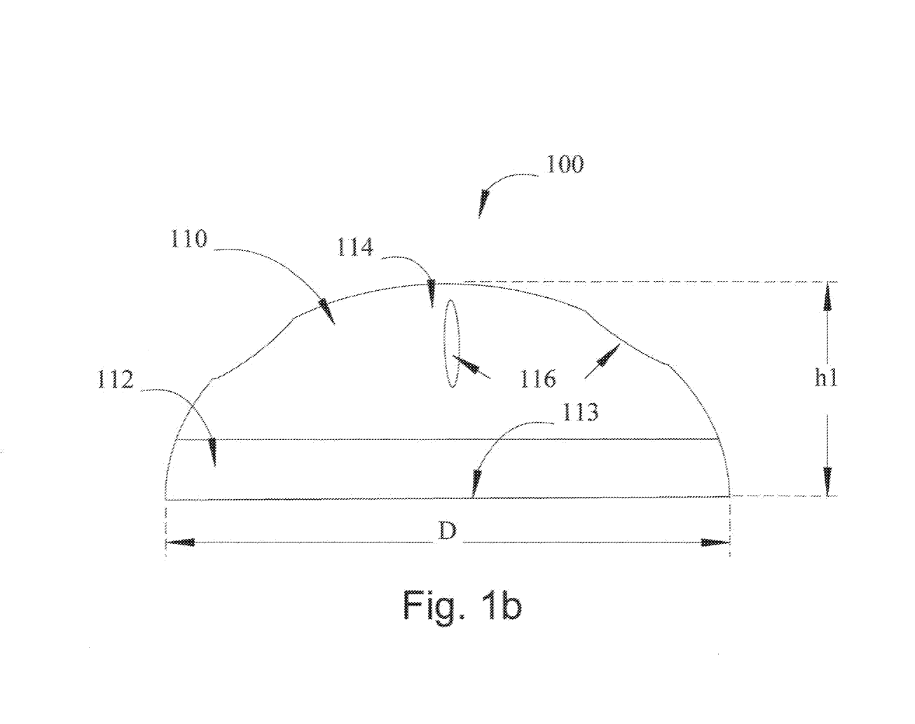

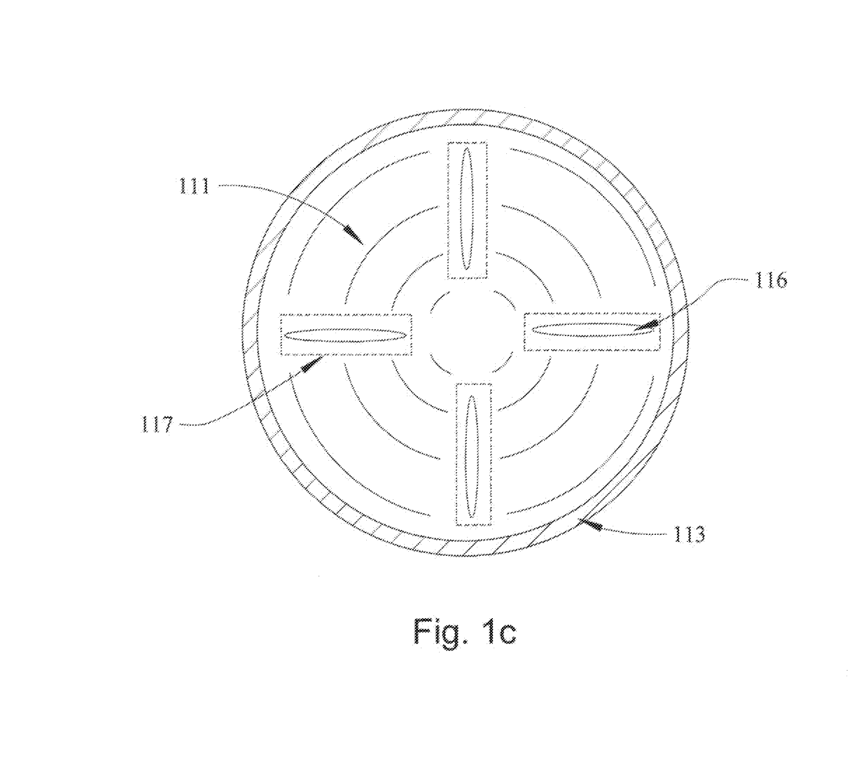

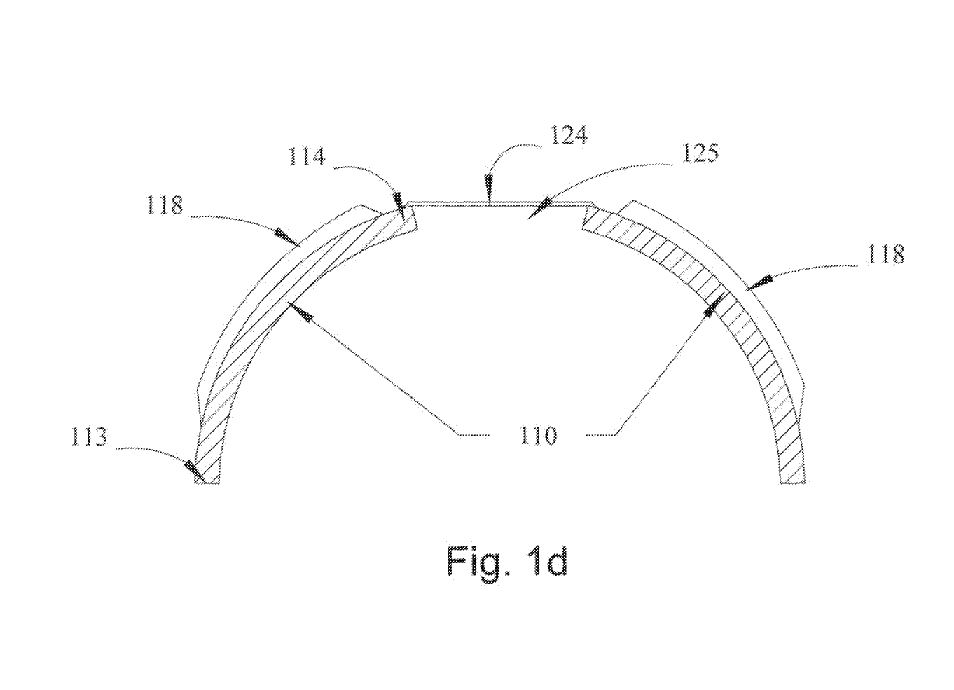

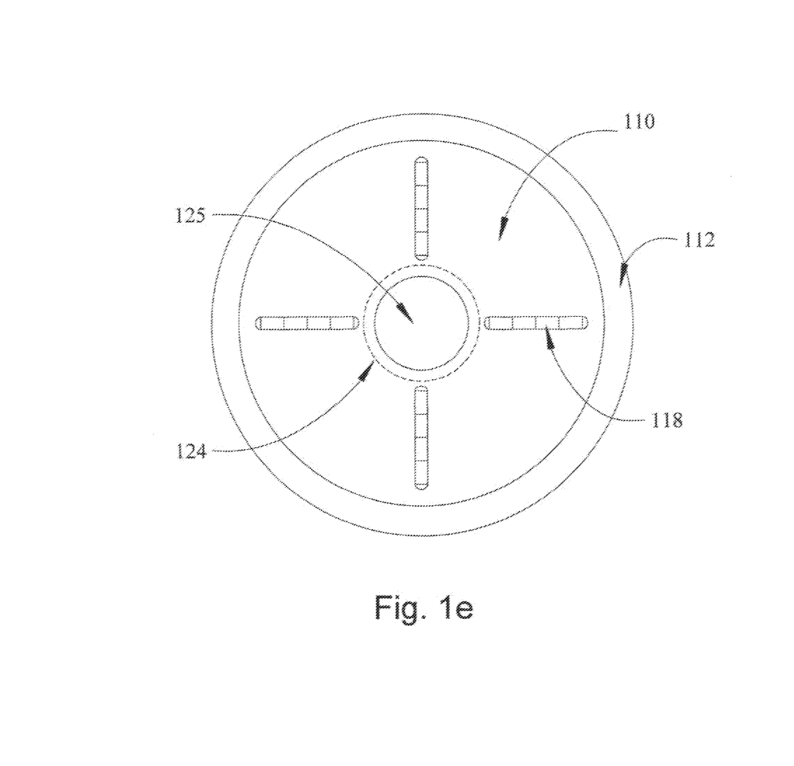

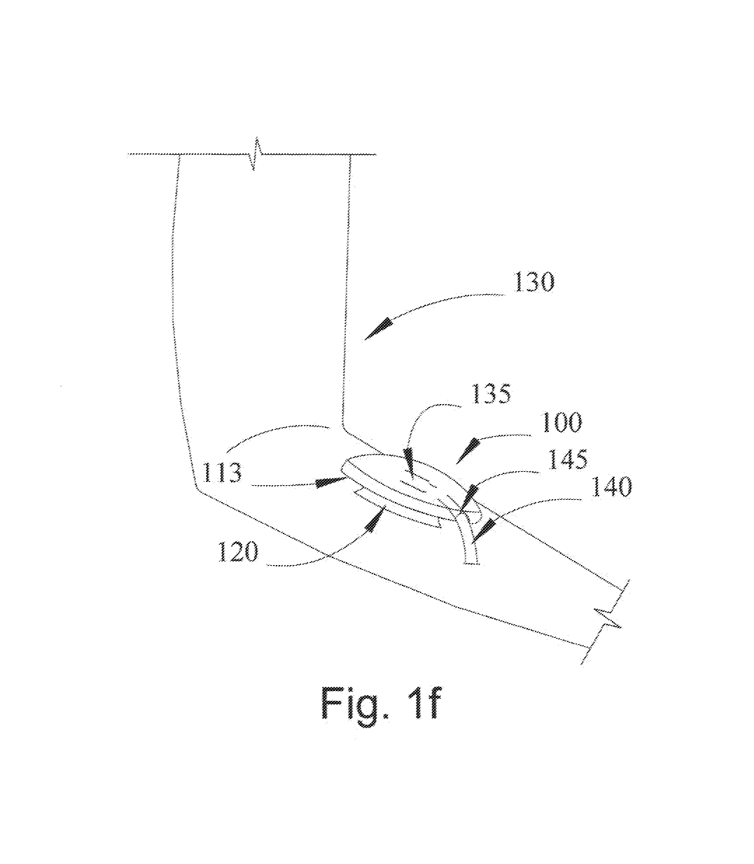

FIGS. 1a-1f illustrate various embodiments of a dome-shaped protective bandage device 100. As depicted in FIG. 1a, the bandage 100 includes a wall 110 that extends radially from a central apex portion 114 to a comfort band 112. In the illustrated embodiment, the wall 110 forms a dome shape with a central apex portion 114 that extends to an outer perimeter or radial edge 113. The outer radial edge 113 and central apex portion 114 define a height dimension h1 (See FIG. 1b). The height h1 can be of any desired dimension but generally ranges from about 0.25 inches to about 5 inches; alternatively from about 0.5 inches to about 3 inches. The wall 110 has a generally hemispherical or semispherical shape. As illustrated in FIG. 1c, the hemispherical shape defines a concave interior portion 111. Other shapes of wall 110 can include circular, polygonal, or an elliptical shape. Preferably, the wall 110 defines a sloping surface that is not perpendicular at the junction with the outer radial edge 113. That is, preferably the angle at the junction between the wall 110 and the outer radial edge 113 is less than 90°, for example from about 10° to about 80°; preferably from about 20° to about 50°. The circular dome shape for wall 110 is beneficial in that as a force is applied to the bandage 100, the force is dissipated equally in all directions. Moreover, the dome shape of wall 110, while accommodating any part of the body, is also useful in covering a wound on curved body parts such as, an elbow, shoulder, knee, ankle, and heel, to name a few. The wall 110 is configured to cover and protect a wound without contacting the wound itself. The comfort band 112 extends to and terminates at the outer perimeter edge 113 and contacts the skin surrounding the wound. In other words, wall 110 forms a bubble or shield of protection over a wound. Moreover, wall 110 can keep the wound in a clean, and if desired, sterile environment.

In one embodiment, the wall 110 of the dome-shaped bandage provides a substantially continuous surface. In another embodiment of the dome-shaped bandage, wall 110 provides at least one hole or vent of any desired size and/or shape to facilitate air circulation during use of the device by an individual in need thereof. As illustrated in FIG. 1c, the holes or vents 116 are preferably covered with a protective material or membrane 117 that allows for air flow while providing a barrier to entry of dust or other environmental debris. Any suitable material can be used for the protective material 117 including natural or synthetic fiber material or mesh material such as cotton or other plant-fiber material, and any suitably porous or breathable plastic material.

The wall 110 can have different sizes to comfortably fit over any desired area of the body to which the bandage 100 is applied. For example, as illustrated in FIG. 1b, wall 110 has a diameter D which can range from about 0.5 inches to about 8 inches. Preferably, diameter D is from about 2 inches to about 4 inches. The dome-shaped embodiment can also have varying height dimension h1 (FIG. 1b); preferably h1 is from about 0.5 inches to about 3 inches; most preferably, h1 is less than or equal to one-half the diameter D.

Wall 110 can be made of various materials preferably materials that are hard or rigid after formation of a shape. In one form, wall 110 is made of any suitable material known to the skilled artisan that is sturdy enough to withstand pressure without collapsing onto a wound. Suitable but non-exclusive materials for wall 110 include various synthetic or semi-synthetic plastics or thermoplastic materials including but not limited to polystyrene, PVC, nylon, polycarbonate, and acrylic that can be clear, colored, or opaque. For example, one might choose to manufacture wall 110 with an opaque or colored plastic if a person using the bandage does not want to view the wound.

In the illustrated embodiment of FIGS. 1a-1c, wall 110 has a substantially smooth exterior surface; however, in other embodiments, wall 110 can include one or more ridge structures 118 to strengthen wall 110 (See FIGS. 1d and 1e). FIGS. 1d and 1e depict a bandage having four ridges 118 spaced at about 90° intervals. It should be understood that other configurations and arrangements for ridges are within the scope of the invention including bandage devices having from 2 to 8 ridges spaced approximately substantially evenly or unevenly and being spaced at about 45° to about 180° intervals around wall 110. In some embodiments, wall 110 can include pictures or illustrations for decoration or entertainment, for example, when the device is to be used by children.

In the illustrated embodiment of FIG. 1a, comfort band 112 forms a ring or circular shaped structure around the wall 110. In other embodiments, comfort band 112 can have another shape such as oval, elliptical, polygonal, or other configuration to engage the wall 110 and/or as medically required. The comfort band 112 can be made of the same material or a different material as the wall 110. The comfort band 112 can have the same or different material properties as wall 110. For example, wall 110 can be made of a hard, rigid plastic material while comfort band 112 is made of a soft, pliable plastic material. In this form, comfort band 112 would feel soft as it is placed on a person's skin around a wound. Ideally, comfort band 112 is sized larger than a wound so as not to contact or rest against the wound.

In the illustrated embodiment of FIGS. 1a and 1b, the wall 110 includes four vents 116 wherein each of vents 116 is spaced about 90° (90 degrees) from an adjacent vent around the central portion 114. In other embodiments, wall 110 can include a greater or lesser number of vents 116 or none at all. The vents 116 are beneficial in allowing air flow and moisture escape from the wound protective bandage 100 when the bandage 100 is applied over a wound or ancillary medical device. As shown in FIG. 1c, in a preferred embodiment the vents 116 are covered by a covering material or membrane 117 that allows free air flow while blocking entry of environmental debris, dust, or other material. The covering material 117 may be placed over the vents 116 on the top side of the dome, or, as illustrated in FIG. 1c, on the underside of the dome. While FIG. 1c shows the covering material 117 to be approximately the same size and shape as the vents 116, other configurations and/or shapes can be used. For example, the covering material 117 could comprise a single piece of material that covered one or a plurality, including all, vents 116.

In another variation of a bandage of the invention, as illustrated in FIGS. 1d and 1e, the wall 110 includes a central aperture 125 at the central apex portion 114. The central aperture 125 can be of any suitable and/or desired size, shape, and dimension to facilitate access to a wound or ancillary medical device without having to dislodge and remove the bandage from the patient, for example, when applying a medication to a wound or when checking or servicing an ancillary medical device. In a preferred embodiment, central aperture 125 has a diameter of from about 0.5 inches to about 3 inches and is covered by a protective membrane 124 that can be soft and/or pliable. The protective membrane 124 can be lifted as needed to expose and provide easy access to a wound or ancillary medical device without disturbing the bandage. Alternatively, central aperture 125 can have a hard and/or rigid covering or closure means that may be opened or closed for easy access to a wound. Alternatively still, central aperture 125 can be left open without any covering. The protective membrane can be made from any suitable material including but not limited to natural or synthetic fiber mesh material, gauze, plastic, thermoplastic, etc. Preferably, the membrane 124 has an adhesive applied, for example, around the edge of membrane 124 to facilitate repeated lifting and replacing the membrane onto the bandage as desired.

The bandage 100 also preferably includes attachment means by which to secure the device to a patient. Any suitable attachment means can be used including, but not limited to, adhesives that are applied to the skin, adhesive strips, tape, wraps, etc. FIG. 1a illustrates one embodiment in which adhesive tabs 120 are connected to a portion of the wall 110 and/or the comfort band 112 wherein the adhesive tabs 120 are configured to attach to the skin surrounding a wound or ancillary medical device until it is desired to remove the tabs 120 (FIG. 1a). Each of the adhesive tabs 120 is spaced around the central portion 114 at about 90° from an adjacent adhesive tab 120. Other embodiments can include a greater or lesser number of adhesive tabs 120 wherein the adhesive tabs 120 would be spaced around the central portion 114 in a spaced-apart relation to the wound. In yet another embodiment, a continuous adhesive tab can be applied along the comfort band 112 or outer edge 113. In the illustrated embodiment, the adhesive tabs 120 have a generally rectangular shape and are sized to fit around the bandage. In other embodiments, the adhesive tabs 120 can also have another shape and/or size. The adhesive tabs 120 can have a different size and/or shape from one another. Additionally, as illustrated in FIG. 1a, each of the adhesive tabs 120 may include a relief slit 122 that facilitates the adhesive tab 120 to lay flat against a person's skin when it is applied. In other embodiments, an adhesive tab 120 is continuous, that is without the relief slit 122.

In another variation of the dome-shaped embodiment, one or more ancillary medical device passages or holes are located on the top portion for the accommodation of ancillary medical devices. In a preferred embodiment, the one or more passages are located near or around the radial edge. Such passage(s) are particularly useful in a hospital or institutional environment when the device is used to protect an ancillary medical device that may include tube(s) that emerge from the site of placement. The ancillary medical device passages of this type provide an outlet for such tubes that avoids disturbing the secure placement of the device on the patient. FIG. 1f illustrates placement of a dome-shaped device 100 on the arm 130 of a patient. As illustrated, the device 100 is placed over an ancillary medical device 135 from which a tube 140 passes through an ancillary medical device passage hole 145 located at the outer edge 113 of the device.

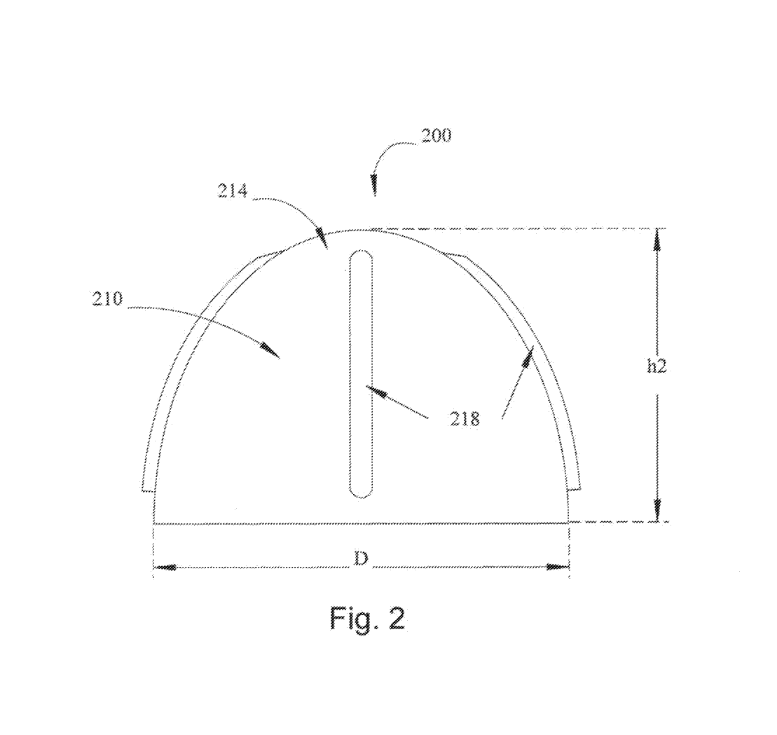

FIG. 2 illustrates a second embodiment of a wound protective bandage 200 according to the present invention. Bandage 200 is similar to bandage 100; therefore, for the sake of brevity similar features will not be extensively described. Bandage 200 also includes a wall 210. Wall 210 forms a bowl shape with a central portion 214. In the illustrated embodiment, wall 210 includes a plurality of ridges 218 to strengthen the wall 210. In other embodiments, wall 210 includes a single ridge or is smooth and lacks ridges. As with the first embodiment, the second embodiment may also include one or more vents to facilitate air flow and/or a central aperture. Wall 210 can assume any size to fit over a wound but not contact or touch the wound. For example, the embodiment of FIG. 2 can have a diameter D of from about 1 inch to about 6 inches. The configuration, shape, and size of wall 210 may be beneficial for positioning bandage 200 over a wound or ancillary medical device on a part of the body that is not flat or smooth, for example, a knee or elbow. As with the first embodiment (FIGS. 1a-1c), bandage 200 includes a height dimension h2. Preferably, h2 is greater than the height dimension h1 of the first embodiment. Height dimension h2 can range from about 1 inch to about 8 inches; preferably, h2 is from about 2 inches to about 5 inches.

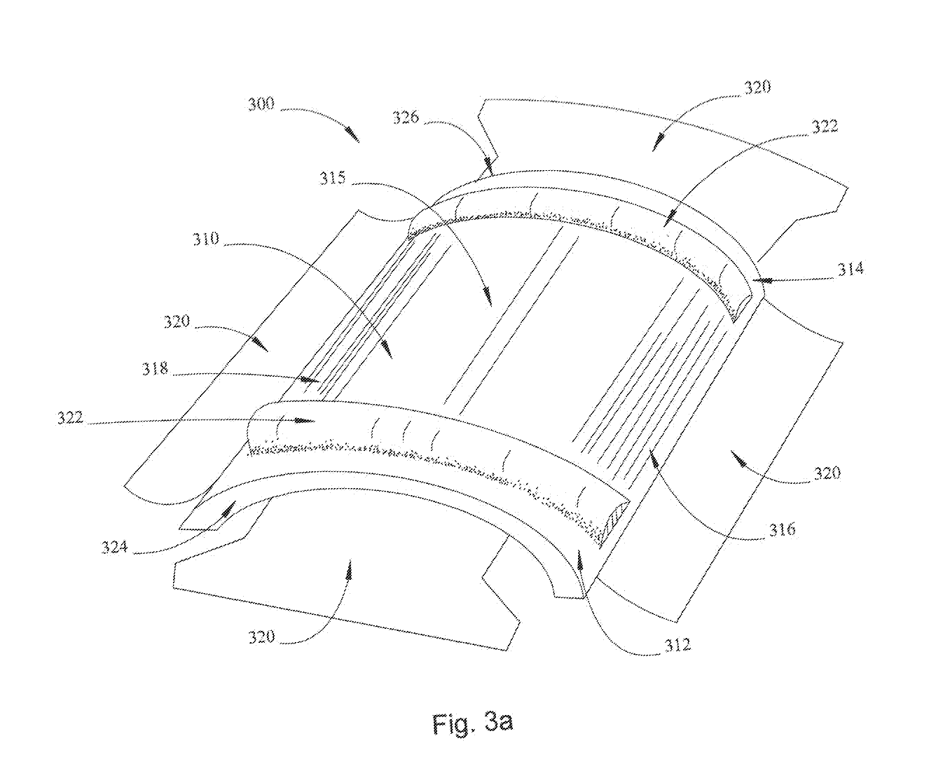

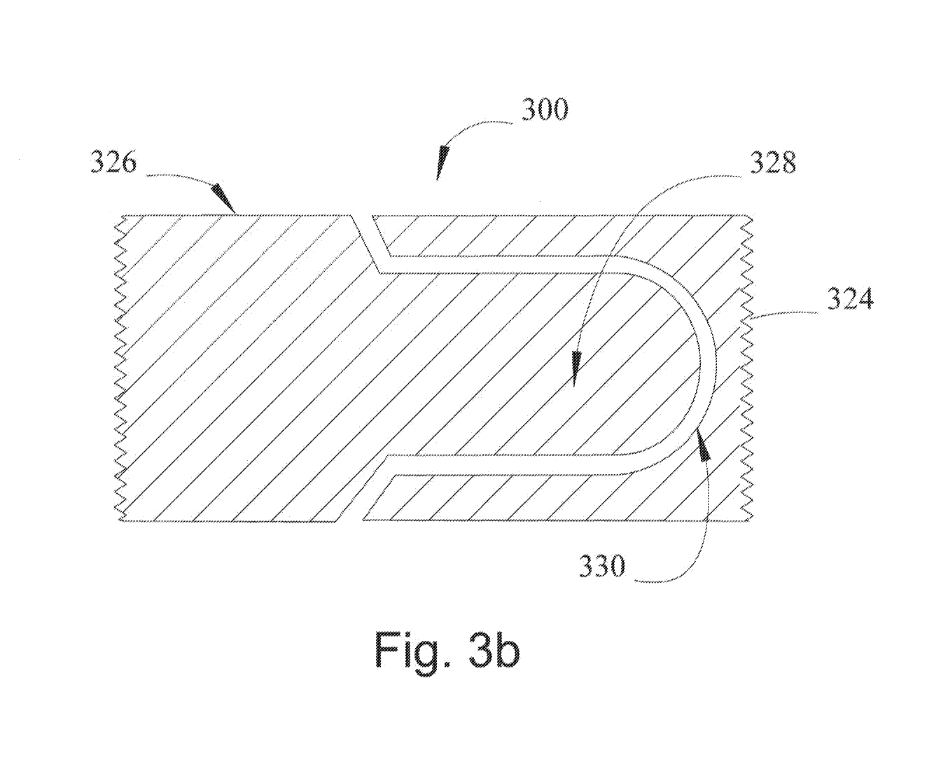

FIGS. 3a and 3b illustrate aspects of a third embodiment of a wound protective bandage of the invention. Bandage 300 includes a wall 310 that extends between a front portion 312 and an opposite a rear portion 314 with a central portion 315 spanning between the front portion 312 and the rear portion 314. The device of the third embodiment includes a front edge portion 324 and a corresponding rear edge portion 326. The wall 310 also includes a right side portion 316 and a left side portion 318. The wall 310 is similar to wall 110; however, wall 310 is configured differently. Wall 310 is similar to wall 110 in that wall 310 is configured to cover or bridge over a wound or ancillary medical device without contacting or touching same. In the illustrated embodiment, wall 310 has a substantially rectangular arch shape. Therefore, when bandage 300 is placed over a wound or ancillary medical device, the right side portion 316 and the left side portion 318 contact the skin near the wound. However, the front portion 312, rear portion 314, and central portion 315 should not contact the wound or ancillary medical device if the bandage 300 is sized and positioned properly on the user. As such, wall 310 can be manufactured in any range of sizes to fit the desired body area and wound to which the bandage 300 is applied. For example, in the illustrated embodiment, wall 310 has a length of about 3 to 4 inches and a width of about 2½ inches. Wall 310 generally has an arch shape but can be other than rectangular such as, for example, square or other polygonal arched shape. Similar to bandage 100, bandage 300 can also be used to cover or bridge over an ancillary medical device, for example, a peripherally inserted central catheter, an intravenous catheter, stitches, splint, stent, syringe, or other medical device inserted into or onto the body wherein bandage 300 is applied at the point of entry or attachment to the body.

The front portion 312 and the rear portion 314 can be configured in a variety of ways to enable a reversible or non-reversible coupling between two or more bandages. In a preferred embodiment the configuration enables a reversible coupling of a plurality of bandages. In this aspect, the embodiments include any suitable means known to the skilled artisan for reversibly coupling mechanical parts, in particular means to couple or connect bandages of the present invention. Suitable coupling means include, but are not limited to, interlocking means such as male-female type connections, adhesive couplings, and connections using Velcro. In one embodiment, the front portion 312 and rear portion 312 can be configured to form an interlocking mechanism with another bandage having corresponding mating front and rear portions 312 and 314. For example, as shown in FIG. 3a, a first bandage 300 has at the front portion 312 and rear portion 314 connecting ridge-like structures 322 that enable reversibly connecting two or more bandages 300 in an end-to-end fashion by providing male and female-type structures to enable a reversible connection. In one embodiment, as depicted in FIG. 3a, connecting ridge-like or ridge-shaped structures 322 are configured to have an outer convex surface and a hollow concave interior region. The concave interior region is configured to receive the outer convex surface of another bandage 300 thereby coupling two bandages together. For example, the front portion 312 of one bandage comprises a female receiving end and the rear portion 314 of a second bandage 300 comprises a male end that is inserted into the front portion 312 of the first bandage 300 to form a reversibly interlocked pair between the first and second bandages 300. Any desired number of bandages 300 can be attached together to form a continuous line of bandages 300 to cover larger wounds or one or more ancillary medical devices.

Wall 310 can be made of various materials that are preferably hard or rigid after formation of a desired shape. In one form, wall 310 is made of any material that is sturdy enough to withstand constant pressure without collapsing onto the wound. Moreover, wall 310 is preferably sturdy or rigid enough that when pressure is applied to the wall 310, none of the front portion 312, rear portion 314, central portion 315, right side portion 316, or left side portion 318 will deflect to contact the wound. One example material for wall 310 includes any suitable plastic or thermoplastic material that can be clear, colored, or opaque. As with the dome-shaped embodiment, in some embodiments of an arch-shaped device, wall 310 includes one or a plurality of structural stability ridges to enhance the strength of the bandage. In other embodiments, wall 310 has a substantially smooth surface (See FIG. 3a). Moreover, wall 310 can include pictures or illustrations for decoration.

The bandage 300 also includes one or more adhesive tabs 320. FIG. 3a depicts a device with four adhesive tabs 320 wherein one of tabs 320 is connected to each of the front portion 312, rear portion 314, right side portion 316, and left side portion 318. Adhesive tabs 320 are similar to adhesive tabs 120 and are configured to attach to the skin surrounding a wound or medical device until it is desired to remove the bandage 300. In the illustrated embodiment, the adhesive tabs 320 have a generally rectangular or polygonal shape. The adhesive tabs 320 can be of any suitable size sufficient to securely attach a bandage 300 to a patient. For example, adhesive tabs 320 can range in size from about 0.5 inches to about 5 inches in length, and from about 0.5 inches to about 2 to 3 inches in width. In other embodiments, the adhesive tabs 320 can have another shape and/or size. Moreover, the adhesive tabs 320 on one bandage 300 can have a different size and/or shape from one another.

FIG. 3b depicts another embodiment for bandage 300 showing a partial cross-sectional view of the rear edge 326 and front edge portions 324 of two interlocking bandages 300 to illustrate another means for reversible connecting two or more bandages 300. In this alternative embodiment, the interlocking ridges 322 of the embodiment of FIG. 3a are replaced by interlocking male-female structures at the edges of bandage 300. A rear edge portion 326 of one bandage 300 has a male structure 328 that interlocks with the front edge portion 324 of another bandage 300 having a female structure 330.

### METHODS OF USE

In another aspect, the present invention relates to methods of using a bandage device of the invention to provide comfort to a patient and to promote healing of a wound, whether inflicted surgically or by other means, for example, accidently or during sporting activities. Use of a protective device of the present invention is expected to facilitate the natural healing process by reducing the risk of environmental insults that otherwise might retard the healing process not to mention inflict additional pain and/or discomfort to a patient. A protective device of the present invention is also expected to facilitate comfort to patients and overall clinical benefit to the patient whose treatment involves use of ancillary medical devices such as stents, tubes, splints, syringes, catheters, etc. Such ancillary medical devices often protrude from a patient's body or skin, and are easily disrupted or dislodged thereby impeding the purpose for which they are utilized and possibly inflicting needless additional pain to a patient.

One embodiment of this aspect of the invention relates to a method for treating a wound comprising attaching one or more bandage devices of the present invention to a patient in need thereof. In another embodiment of this aspect, the invention relates to a method for protecting one or more ancillary medical device(s) attached to a patient from disruption or dislodgement comprising covering said ancillary medical device(s) with one or more bandages of the present invention. The methods according to this aspect of the invention are expected to promote healing, for example, by protecting a wound and/or ancillary medical device attached to a patient from environmental stresses or insults including, for example, forces such as bumping, scraping, pressure, etc. that otherwise could impede either the natural healing process or the expected clinical benefit from the ancillary medical device. In addition, the methods of using a bandage of the present invention are expected to reduce pain and discomfort to a patient at least for the reason of protecting against the aforementioned environmental insults typically encountered in everyday life or in a hospital environment.

A device of the present invention can be used by medical and or veterinary professionals in or out of a hospital environment, or by non-medical professionals for home use. For use by medical professionals, including doctors, veterinarians, and nurses as well as other medical care professionals, a device of the invention can be used for any type of wound protection and/or protecting any type of ancillary medical device that may be used on a human or veterinary patient. For example, in a hospital or institutional environment, including nursing homes, a device of the invention can be attached to a patient to protect any type of vessel or tube that is inserted into or otherwise applied to a patient's body for any purpose including, but not limited to nutrition, hygiene, and therapeutic purposes including feeding tubes, syringes, catheters, and the like.

The methods according to this aspect of the invention can be applied alone or in combination with other treatments. In one aspect, a bandage of the present invention is applied immediately after an individual sustains a wound or injury, or soon thereafter, or has an ancillary medical device attached with or without other treatments. For example, in one embodiment a bandage of the invention is attached to a wound without concurrent other treatment(s) or treatment modes. In another embodiment, a bandage of the invention is applied to a wound, including a bleeding wound, that is concurrently or sequentially dressed with another treatment device, for example, a band-aid, absorbent pad, gauze pad, splint, etc. In some aspects, a bandage of the invention is used in conjunction with other therapeutic or pharmaceutical treatments including topically applied or orally administered medications, such as, but not limited to, antibiotics, antiseptics, anti-inflammatory agents, pain reducers, etc.

In another embodiment of this aspect of the invention, a bandage of the invention is used concurrently with, or in succession, or sequentially with other treatments. For example, a wound or ancillary medical device may be initially treated or managed by conventional means, for example, by application of a splint or absorbent pad to absorb blood flow, followed thereafter by application of a protective bandage of the present invention. For example, on day 0, a fracture or bleeding injury is sustained by a patient and a split or absorbent pad is applied as appropriate to the injury. A device of the present invention can be applied in conjunction with such other treatments, for example, on day 0, day 1, day 2, day 3, day 4, day 5, day 6, day 7, day 8, day 9, day 10 or any time thereafter the injury is sustained, as deemed appropriate by the patient or care-giver. In certain cases, it may be advantageous to apply a protective bandage of the present invention on day 1 or later after the injury has occurred, for example, to allow for the control of blood flow.

In some embodiments, bandages are applied to a patient by attaching the bandage to the appropriate site on the patient using adhesive strips or any other means for attachment including but not limited to tape, gauze wrappings, etc. Bandage devices having a centrally located aperture in the wall are especially convenient in cases in which a wound must be dressed during the healing process. The central aperture allows access to the wound without having to lift and remove the bandage device from the patient which may cause pain and/or disruption to the healing process.

While the invention has been illustrated and described in detail in the drawings and foregoing description, the same is to be considered as illustrative and not restrictive in character, it being understood that only preferred embodiments have been shown and described. All changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A protective bandage device comprising:
a. a substantially rigid top portion comprising
i. an inner and outer surface, said inner surface defining a hollow concave chamber between said top portion and the surface of the skin of a person on whom the device is placed;
ii. a sloping wall portion;
iii. an outer perimeter portion which makes contact with the skin of said person; and
wherein said device does not include an absorbent pad.

2. A bandage device as is claim 1 wherein said wall portion comprises a comfort band.

3. A bandage device as in claim 2 wherein said device is dome-shaped or bowl-shaped.

4. A bandage device as in claim 3 wherein the top portion of said device has at least one ventilation opening.

5. A bandage device as in claim 1 wherein said top portion comprises at least one ridge.

6. A bandage device as in claim 5 wherein said top portion further comprises a central aperture.

7. A protective bandage device comprising a wall portion that extends between a front portion and rear portion, said wall portion defining an arch-shaped structure having a convex exterior surface and a concave interior surface, wherein said front portion and rear portion include means for reversibly connecting two or more bandages together.

8. A dome-shaped protective bandage device comprising:
a. a substantially rigid top portion wherein said top portion comprises
i. an inner and outer surface, said inner surface defining a hollow concave chamber between said top portion and the surface of the skin of a person on whom the device is placed;
ii. a sloping wall portion that terminates in an outer perimeter portion wherein the angle between said wall portion and said outer perimeter is less than 90°;
iii. at least one ventilation opening;
iv. at least one ridge;
v. a centrally located aperture; and
wherein said device does not include an absorbent pad.

9. A method of protecting a wound or ancillary medical device on a person in need thereof comprising applying to a patient in need thereof a device of claim 8.

10. A method as in claim 9 wherein said bandage device is applied to a wound substantially simultaneously with other treatments selected from the group consisting of conventional bandages, gauze pads, topical medicaments, antibiotics, anti-inflammatory agents, and pain reducers

11. A method as in claim 9 wherein more than one bandage device is applied.

12. A method as in claim 9 wherein said device is applied after a wound is sustained.

13. A method as in claim 12 wherein said bandage device is applied on from day 1 to day 10 after said wound is sustained.
